(19) United States
(12) Patent Application Publication
(10) Pub. No.: US 2018/0000836 A1
(43) Pub. Date: Jan. 4, 2018

(54) Topical Treatment for Psoriasis

(71) Applicant: Yousif Kattoula, El Cajon, CA (US)

(72) Inventor: Yousif Kattoula, El Cajon, CA (US)

(21) Appl. No.: 15/200,766

(22) Filed: Jul. 1, 2016

(51) Int. Cl. A61K 31/573; A61K 31/555; A61K 9/00; A61K 9/06; A61K 47/34; A61K 31/714

(52) U.S. Cl. CPC A61K 31/573; A61K 47/34; A61K 31/714; A61K 9/0014; A61K 9/06; A61K 31/555

(57) ABSTRACT

A topical skin preparation configured for the treatment of psoriasis, is provided. The preparation includes a pharmaceutically effective amount of clobetasol propionate and other preparation components which work in synergy to significantly enhance the therapeutic effect of the preparation.

### BACKGROUND OF THE INVENTION

### 1. Field of the Invention

The present invention relates to the treatment of patients who suffer from psoriasis. More particularly, the disclosed treatment relates to a topical steroid treatment for direct application to the skin of patients suffering from psoriasis, which includes secondary ingredients yielding a synergistic effect resulting in enhanced effectiveness of the active ingredient thereby accelerating patient treatment.

### 2. Prior Art

Psoriasis is a chronic, noninfectious disease that affects mainly the skin of afflicted patients. While there are many theories on the cause, it is currently suspected to be a disease which is primarily autoimmune in origin.

In patients afflicted with the disease, it commonly causes red, scaly patches to appear on the skin of most patients. Because of the appearance of the disease on the skin of patients, many patients are embarrassed by the symptoms. The scaly patches caused by psoriasis, called psoriatic plaques, are areas of inflammation and excessive skin production. Skin rapidly accumulates at these sites and takes on a silvery-white appearance and subsequent or concurrent inflamation of skin areas. Plaques frequently occur on the skin of the elbows and knees, but can affect any area including the scalp, palms of hands and soles of feet, and even the genitals.

Unfortunately for patients suffering from psoriasis, the disorder is a chronic recurring condition that varies in severity from minor localized patches to complete body coverage. Further, in addition to the rashes and skin discoloration caused by outbreaks of psoriasis, patients in addition to suffering from skin inflamation, also suffer from itching and plaque formation.

Additionally, a majority of patients suffering from psoriasis also suffer psychological issues. Studies have show that psoriasis patients can have feelings of isolation and being excluded due to their condition. There is also medical evidence showing that worrying about psoriasis may make treatment less effective to patients. Further, a 2009 National Psoriasis Foundation survey found that 63% of respondents said their condition affected their emotional well-being, thus showing that psoriasis patients suffer from more than just a skin condition.

The cause of psoriasis is not exact, but it is believed to have a genetic component and it can be triggered by a prolonged injury to the skin. Factors that may aggravate psoriasis are thought to include stress, withdrawal of systemic corticosteroid, excessive alcohol consumption, and smoking.

While there are many treatments available, which range from homeopathic to prescription medications, because of the chronic recurrent nature psoriasis, and the differing nature of patients and their symptoms, is a challenge to treat. Current prescription products available generally employ a single steroid ingredient in the formulation. Because of this and the differing nature of patient symptoms and their differing reactions to medications, frequently they fail to work when treating patients. Further, many other medications are ingested, causing the patient to have to treat the entire body, from localized skin areas afflicted.

Current topical treatments which are prescribed to treat psoriasis, employ topical corticosteroids which are potent anti-inflammatory drugs. While such corticosteroids reduce cell turnover by suppressing the immune system which reduces inflammation, thereby helping to relieve associated itching and inflammation, a high concentration and long term use of such corticosteroids, can cause problems for patients such as a thinning of the skin.

As such, there is a continuing and unmet need for an external and topically administered treatment for psoriasis which may be directly applied only to areas of skin affected. Further, such a topical medication should include a plurality of medications as well as vitamins and the like to provide a broader more effective treatment which is easily targeted by the patient to skin areas, and which will enhance and accelerate results, thereby reducing the term of use of the treatment.

### SUMMARY OF THE INVENTION

The treatment herein, overcomes the noted shortcomings of current treatments for psoriasis, through the provision of topically applied preparation, which is a combination of a topical corticosteroid in with vitamins and minerals. This combination through experimentation, has been found to have a synergistic effect in treatment of psoriasis, where the combination of the active ingredient of corticosteroids with the other noted ingredients augment and potentiate the effect of corticosteroids at the cellular level, thereby significantly improving the outcome of psoriasis therapy resulting in a faster response time, and less steroids being employed by the patient, thereby lessening the potential negative affects of such use.

The medication herein employs a formulation which is composed of clobetasol propionate 0.01-1.0%, cyanocobalamin (Vitamin B12) 0.01-2.0%, zinc pyrithione 0.01-2.0%, 1-50% of silicone base gel, 1-50% of a lipophilic base cream, and 1-50% hydrophilic base.

In experimentation, a significant improvement has been found in the therapeutic effect of the active ingredient of clobetasol propionate, with the addition of Vitamin B12, as well as zinc in the noted percent ranges, which is included with the base including one or a combination of base ingredients from a group including Silicone base gel, lipophilic base cream, and a hydrophilic base. The synergistic effect of the combined ingredients provided a significant improvement in accelerating treatment of plaque psoriasis patients using the combination of ingredients over that of the single active ingredient alone.

The topical application using the preparation herein for treatment of psoriasis according to the present invention, to accelerate healing, merely requires a once to twice daily application of the formed preparation to the affected areas for a period of six to twelve weeks. Generally, no special precautions are required other than the general precautions which accompany the topical application of topical steroids to the skin. Evaluation of the effectiveness of the preparation herein, is by simple examination of the patient and reduction of rash and other skin symptoms, and by changes in the amounts of itching present.

Topical application of the preparation for plaque psoriasis treatment herein to affected skin areas, on a twice, daily basis, generally produces beneficial effects on plaque psoriasis when applied over a period of time between six to twelve weeks. The treatment has been highly successful in substantially every case with complete or substantially complete clearing of psoriasis affected areas. The clearing noted lasting for a prolonged period after treatment.

### EXAMPLE 1

Compared to commercially available skin formulations which had been ineffective, the results based upon the efficacy of the present invention are set forth hereinafter: A female patient was bothered by frequent plaque psoriasis on both her elbows, both her knees, her earlobes and behind her ear, which resulted in the normal physical discomfort as well as the rash and redness attributable to plaque psoriasis. Several conventional therapies had been tried for years without success, other than a minor reduction in symptoms.

The product herein made was provided the patient and was applied by the patient in a thin layer twice a day to all affected areas. All of affected sites with plaque psoriasis were totally cleared within 8 to 12 weeks.

With respect to the above description, before explaining preferred embodiments of the topical preparation herein in detail, it is to be understood that the invention herein is not limited in its application to the details of formulation and to the arrangement of the components in the following description or as illustrated in the drawings. The invention herein described is capable of other embodiments and of being practiced and carried out in various ways which will be obvious to those skilled in the art upon reading this disclosure. Also, it is to be understood that the phraseology and terminology employed herein are for the purpose of description and should not be regarded as limiting.

As such, those skilled in the art will appreciate that the conception upon which this disclosure is based, may readily be utilized as a basis for formulating other similar topical treatments for psoriasis and for carrying out the several purposes of the present disclosed device. It is important, therefore, that the claims herein be regarded as including such equivalent construction and methodology insofar as they do not depart from the spirit and scope of the present invention.

It is an object of the invention to provide a highly effective topical preparation for treatment of psoriasis.

It is another object of this invention to provide such a topical preparation for psoriasis which when applied, significantly accelerates healing.

It is another object of the invention to provide such a topical cream treatment, which includes secondary ingredients inducing an enhanced synergistic therapeutic effect of the corticosteroid component of the topically applied treatment.

Further objects of the psoriasis treatment preparation will be brought out in the following part of the specification, wherein detailed description is provided for the purpose of fully disclosing the invention but without placing limitations thereon.

### BRIEF DESCRIPTION OF DRAWING FIGURES

FIG. 1 depicts the ingredients of the formed topical preparation yielding accelerated healing of psoriasis.

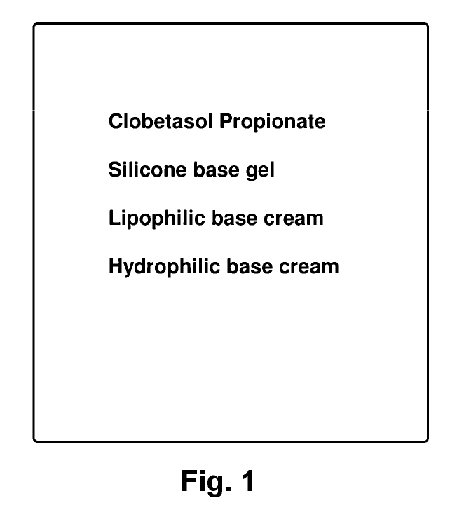

### DETAILED DESCRIPTION OF THE PREFERRED EMBODIMENTS OF THE INVENTION

In the practice of the skin preparation of the present invention, clobetasol propionate is distributed according to known techniques in various pharmaceutically acceptable carriers such as emulsions, solutions, suspensions including lotions, creams and ointments. Some of these carriers may contain wetting agents, emulsifying and suspending agents.

In one mode of preparation herein it includes a pharmaceutically effective amount of clobetasol propionate, in a combination with secondary ingredients of cyanocobalamin (Vitamin B12), and zinc pyrithione. While the clobetasol propionate alone in a pharmaceutically effective amount when combined with the carrier such as a combination of a silicone base gel, lipophilic base cream, and a hydrophilic base, yielded a treatment providing a reduction or elimination of the plaque psoriasis when applied thereto, the inclusion of the secondary ingredients appear from patient results to provide a synergistic action in combination with the clobetasol propionate in the preparation, yielding an enhanced result over clobetasol propionate alone. The preparation in either mode additionally includes a silicone base gel, a lipophilic base cream, and a hydrophilic base.

In a particularly preferred composition of the preparation herein, for topical treatment of plaque psoriasis, the formulation is composed in percentages by total weight of the mixed formulation of clobetasol propionate 0.01-1.0%, cyanocobalamin (Vitamin B12) 0.01-2.0%, zinc pyrithione 0.01-2.0%, and 1-50% of silicone base gel, 1-50% of a lipophilic base cream, and 1-50% hydrophilic base. In this formulation of the preparation herein, a significant improvement has been found in the therapeutic effect of the primary active ingredient of clobetasol propionate.

This synergistic effect of the secondary ingredients of cyanocobalamin, and zinc pyrithione with the clobetasol propionate, in the preparation applied to affected areas by patients over an eight week duration, provided a significant improvement in accelerating treatment of plaque psoriasis, over clobetasol propionate alone, and a treatment which eliminated the plaque psoriasis where conventional treatments noted below failed.

The outcome of treatment employing the preferred composition of the preparation herein, provided patients with full relief from symptoms of plaque psoriasis in about eight weeks time, while over the counter topical treatments containing salicylic acid and coal tar failed. The preparation herein also outperformed topical treatments containing substances such as aloe vera, jojoba, zinc pyrithione and capsaicin, and topical steroids which are widely and ineffectively used to moisturize, soothe, remove scale or relieve itching.

In a regimen for treatment of plaque psoriasis patients, the preparation herein is mixed and provided to the patient for topical application twice daily for a period of six to twelve weeks, depending on the individual patient results which may be evaluated after each four to six week period by an examination of the areas of body of the patient, and the reduction or total clearing of rash and other skin symptoms in those area. Should symptoms reappear after a clearing, topical application of the preparation herein may be restarted.

In another mode of the preparation herein, the preparation included clobetasol propionate 0.01-1.0%, cyanocobalamin (Vitamin B12) 0.01-2.0%, zinc pyrithione 0.01-2.0%, in a base formed of 20% of silicone base gel, 40% of a lipophilic base cream, and 20% hydrophilic base. Other noted mixtures of ingredients can also employ a base formed of 20% of silicone base gel, 40% of a lipophilic base cream, and 20% hydrophilic base.

In a method of treatment of a patient having plaque psoriasis the preparation in any of the noted preferred modes herein, is provided to the patient in a sealable container. The patient is instructed to apply the preparation to the affected areas twice daily until the symptoms clear.

While all of the fundamental characteristics and features of the preparation for accelerated treatment of psoriasis, have been shown and described herein, with reference to particular embodiments thereof, a latitude of modification, various changes and substitutions are intended in the foregoing disclosure and it will be apparent that in some instances, some components of the preparation may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions to the disclosed preparation for treatment of psoriasis, are included within the scope of the invention as defined by the following claims.

What is claimed:

1. A topical skin preparation adapted-to-the treatment of psoriasis, the preparation comprising:
a pharmaceutically effective amount of clobetasol propionate, mixed with:
a silicone base gel in an amount between 1.0% and 50% of the preparation by weight;
lipophilic base cream in an amount between 1.0% and 50% of the preparation by weight; and
hydrophilic base cream, in an amount between 1.0% and 50% of the preparation by weight.

2. The topical skin preparation claim 1 wherein said pharmaceutically effective amount of clobetasol propionate, is an amount of more than 0.01% of the preparation by weight.

3. The topical skin preparation of claim 2 additionally comprising:
cyanocobalamin in an approximate amount of more than 0.01% of the preparation by weight; and
zinc pyrithione in an approximate amount of more than 0.01% of the preparation by weight.

4. The topical skin preparation of claim 2 wherein, said amount of silicone base gel is 20% of the preparation by weight, said amount of lipophilic base cream is 40% of the preparation by weight, and said amount of hydrophilic base is 40% of the preparation by weight.

5. The topical skin preparation of claim 3 wherein, said amount of silicone base gel is 20% of the preparation by weight, said amount of lipophilic base cream is 40% of the preparation by weight, and said amount of hydrophilic base is 40% of the preparation by weight.

6. A method for accelerating healing psoriasis, which comprises topically administering to a patient in need of such acceleration, the topical preparation of claim 3, twice daily for a duration between six to twelve weeks.
